(19) United States
(12) Patent Application Publication
(10) Pub. No.: US 2019/0307469 A1
(43) Pub. Date: Oct. 10, 2019

(54) CRANIOTOME GUARD

(71) Applicants: Michael A. Greminger, Duluth, MN (US); Anastasia N. Zink, Minneapolis, MN (US); Brian J. Krohn, St. Paul, MN (US); Amit Goyai, St. Anthony, MN (US)

(72) Inventors: Michael A. Greminger, Duluth, MN (US); Anastasia N. Zink, Minneapolis, MN (US); Brian J. Krohn, St. Paul, MN (US); Amit Goyai, St. Anthony, MN (US)

(21) Appl. No.: 16/309,395

(22) PCT Filed: Jun. 15, 2017
(86) PCT No.: PCT/US17/37737

§ 371 Date: Dec. 12, 2018

Related U.S. Application Data
(60) Provisional application No. 62/350,490, filed on Jun. 15, 2016.

(51) Int. Cl. A61B 17/17; A61B 17/16; A61B 90/00

(52) U.S. Cl. CPC A61B 17/1739; A61B 17/1695; A61B 17/1615; A61B 2090/036; A61B 2090/08021; A61B 2217/007; A61B 90/03

(57) ABSTRACT

A device includes an elongate leg, a guard, and a fluid discharge nozzle. The elongate leg has a longitudinal axis. The elongate leg has a first end and a second end. The first end is configured to couple with a craniotome wherein the longitudinal axis is aligned substantially parallel with a cutter axis of the craniotome. The guard is coupled to the second end and has a surface configured to engage with a tissue. The guard is configured to preclude contact of tissue with an end of a cutter of the craniotome. The fluid discharge nozzle is coupled to the guard and configured to direct an ejected fluid that targets separation the dura and the cranium ahead of the cutter to remove dura from a cutting path of the cutter.

### CLAIM OF PRIORITY

This patent application claims the benefit of priority of U.S. Provisional Patent Application Ser. No. 62/350,490, filed on Jun. 15, 2016, and which is incorporated herein by reference in its entirety.

### BACKGROUND

A craniotomy is a procedure in which a piece of the skull is removed in order to gain access to the brain. This is commonly done to remove brain tumors, treat epilepsy, and for traumatic brain injury. Currently, the procedure involves drilling one or more burr holes and then using a tool known as a craniotome to complete the cut.

A craniotome includes a high speed rotary cutting tool. A guard can be fitted to the craniotome to prevent contact between the dura and the cutting tool.

A dural tear occurs in approximately 20-30% of craniotomy procedures. Approximately 160,000 craniotomies are performed each year in the United States. A dural tear adds time to the craniotomy procedure due to the increased difficulty in suturing the dura and the potential need to use synthetic dura material in order to reclose the dura. Also, if the dura tears while using the craniotome, the brain is no longer protected as the craniotomy is completed.

### SUMMARY

The present inventors have recognized, among other things, that a problem to be solved can include reducing the incidence of a dural tear. The present subject matter can help provide a solution to this problem, such as by providing a fluid discharge nozzle in a dura guard. According to one example, a craniotome is configured to discharge a pressurized fluid, such as a sterile saline solution, to facilitate separation of the dura from the cranium ahead of the craniotome in order to reduce the risk of the dura snagging on the dura guard.

In various examples, a fluid delivery nozzle can be incorporated in a craniotome equipped with a guard or can be incorporated into a guard that can be fitted to an existing craniotome. The fluid delivery nozzle can be coupled to a fluid channel disposed within the guard such that a pressurized fluid can be transported to the delivery nozzle during operation of the craniotome.

Each of these non-limiting examples can stand on its own, or can be combined in various permutations or combinations with one or more of the other examples.

The present application provides for the following exemplary embodiments or examples, the numbering of which is not to be construed as designating levels of importance:

Example 1 provides a device comprising an adapter configured to couple with a craniotome, an elongate leg having a longitudinal axis, a guard, and a fluid discharge nozzle. The elongate leg can have a first end and a second end, the first end coupled to the adapter, wherein the longitudinal axis is aligned substantially parallel with a cutter axis of the craniotome. The guard can be coupled to the second end and have a surface configured to engage with a tissue and configured to preclude contact of tissue with an end of a cutter of the craniotome. The fluid discharge nozzle can be coupled to the guard and configured to direct an ejected fluid from the guard.

Example 2 provides the device of Example 1 optionally configured such that the fluid discharge nozzle has a discharge axis and wherein an included angle between the discharge axis and the cutter axis is about equal to or less than 90 degrees.

Example 3 provides the device of any one or more of Example 1 or 2 optionally configured such that the nozzle is configured to eject the fluid in a direction aligned radially with respect to the cutter axis.

Example 4 provides the device of any one or more of Examples 1-3 optionally configured such that the fluid discharge nozzle is configured to discharge fluid in a fan pattern.

Example 5 provides the device of any one or more of Examples 1-4 optionally configured such that the elongate leg has a lateral width less than a cutter width of the cutter.

Example 6 provides the device of any one or more of Examples 1-5 optionally configured such that a cutting surface of the cutter is in opposition to the elongate leg.

Example 7 provides the device of any one or more of Examples 1-6 optionally configured such that the fluid discharge nozzle is fluidly coupled to a fluid path disposed within the guard.

Example 8 provides the device of Example 7 optionally configured such that the fluid path is disposed within at least a portion of the elongate leg.

Example 9 provides the device of any one or more of Example 7 or 8 optionally configured such that the fluid path is contained within a tube disposed in an interior of the elongate leg and the guard.

Example 10 provides the device of any one or more of Examples 1-9 optionally further comprising a suction port coupled to the guard.

Example 11 provides the device of Example 10 optionally configured such that the suction port is coupled to a vacuum channel disposed within the guard and the elongate leg.

Example 12 provides the device of any one or more of Examples 1-11 optionally configured such that the adapter includes an opening configured to receive the cutter such that a portion of the cutter passes through the adapter and attaches to the craniotome.

Example 13 provides a device comprising: an adaptor having a mounting surface configured to fixedly couple with a craniotome, the craniotome having a cutter; an elongate leg having a first end coupled to the adapter and a second end distal from the craniotome, the elongate leg having a leg axis aligned substantially parallel with a rotation axis of the cutter, the second end configured to limit exposure of a contact end of the cutter; and a fluid channel at least partially disposed within the elongate leg and coupled to a pressurized fluid source at the first end, the fluid channel coupled to a discharge port. The discharge port is configured to eject a fluid from the fluid channel. The cutter has a working surface relative to the rotation axis and a nonworking surface relative to the rotation axis, wherein the working surface and the nonworking surface are semicircular. The discharge port is disposed proximate the working surface and distal from the non-working surface, and wherein the elongate leg is proximate the non-working surface and distal from the discharge port.

Example 14 provides the device of Example 13 optionally further comprising a foot portion coupled to the second end of the elongate leg and oriented generally traverse to the leg axis, the foot portion configured to limit an insertion depth of the cutter.

Example 15 provides the device of Example 14 optionally configured such that the fluid channel is disposed within the foot portion.

Example 16 provides the device of any one or more of Examples 13-15 optionally further comprising a suction port coupled to a second channel, wherein a portion of the second channel is disposed within the elongate leg.

Example 17 provides the device of any one or more of Examples 13-16 optionally configured such that the discharge port is configured to eject the fluid in a fan pattern.

Example 18 provides a method of assembling a craniotome for performing a craniotomy, the method comprising: providing a guard portion, the guard portion comprising: an adapter having a feature for attachment to a craniotome; a leg having a first end coupled to the adapter and a second end opposite the first end; a foot coupled to the second end of the leg, the foot and leg oriented generally transverse to one another, the foot including a channel extending through an interior of the foot; and a discharge nozzle coupled to the channel and configured to discharge a fluid flowing through the channel; attaching the guard portion to the craniotome; attaching a burr to the craniotome, the burr configured to rotate relative to the adapter during operation of the craniotome; and orienting the burr and the guard portion relative to each other such that the leg and foot restrict exposure of the burr.

Example 19 provides the method of Example 18 optionally configured such that attaching the burr to the craniotome includes passing the burr through an opening in the adapter.

Example 20 provides the method of any one or more of Example 18 or 19 optionally configured such that the fluid flowing through the discharge nozzle has a discharge axis, the method further comprising orienting the burr and the guard portion relative to each other such that an angle between the discharge axis and a cutter axis of the cutter is less than or equal to about 90 degrees.

Example 21 provides the method of any one or more of Examples 18-20 optionally further comprising connecting the channel in the foot to a fluid supply to deliver the fluid through the channel and to the discharge nozzle.

Example 22 provides a method of performing a craniotomy on a patient, the method comprising: moving a rotating cutting tool along a cutting path relative to a cranium of the patient; interposing a guard between the cutting tool and a dura of the patient such that exposure of the cutting tool to the dura is restricted; and ejecting fluid from the guard in a direction aimed at a junction of the dura and the cranium to separate the dura and the cranium and to remove the dura from the cutting path.

Example 23 provides the method of Example 22 optionally configured such that the guard and the cutting tool are part of a craniotome.

Example 24 provides the method of any one or more of Example 22 or 23 optionally configured such that the guard includes an adapter to removably attach the guard to the craniotome.

Example 25 provides the method of any one or more of Examples 22-24 optionally configured such that the guard includes an elongate leg and a foot extending from the elongate leg, the foot oriented generally transverse to the elongate leg.

Example 26 provides the method of any one or more of Examples 22-25 optionally configured such that ejecting fluid from an end of the guard includes coupling a discharge nozzle to an end of the guard.

Example 27 provides the method of Example 26 optionally configured such that the discharge nozzle is configured to eject the fluid at an acute angle relative to a cutter axis of the cutting tool.

Example 28 provides the method of any one or more of Examples 22-27 optionally configured such that ejecting fluid from an end of the guard includes transporting fluid through a channel within an interior of the guard.

Example 29 provides the method of any one or more of Examples 22-28 optionally further comprising supplying pressurized fluid to the channel.

Example 30 provides the device and methods of any one or any combination of Examples 1-29, which can be optionally configured such that all elements recited are available to use or select from.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a craniotome, according to one example.

FIG. 2 illustrates a craniotome, according to one example.

FIG. 3 illustrates a craniotome, according to one example.

FIG. 4 illustrates a section view of a craniotome, according to one example.

FIG. 5 illustrates a craniotome, according to one example.

FIG. 6 illustrates a method associated with a craniotome, according to one example.

### DETAILED DESCRIPTION

FIG. 1 illustrates system 100 including device 200A, according to one example. Device 200A can include a craniotome or can include an adapter that couples to a craniotome. In an example, device 200A can be referred to as a guard portion of the craniotome and such guard portion can include the adapter for removably attaching the components of the guard (described below) to the craniotome. In the example shown, device 200A includes rotary burr or cutter 150. Rotary burr 150 can include a high speed cutting tool having cutting edges configured for cutting bone. Rotary burr 150 has a cutter length suited to cut the thickness of cranium 20. Distal end 152 of burr 150 extends beyond the cranium 20.

Guard 250A is configured to prevent contact between burr 150 and dura 40. Guard 250A is configured to wrap around distal end 152.

Guard 250A includes a discharge nozzle 60 configured to eject fluid 65 at a juncture between cranium 20 and dura 40.

Discharge nozzle 60 can be designed to eject fluid 65 at a pressure sufficient to separate the dura 40 from the skull or cranium 20 without damaging the dura 40. The discharge nozzle 60 can be sized for engagement with a fluid channel formed through a portion of guard 250A (see FIG. 4).

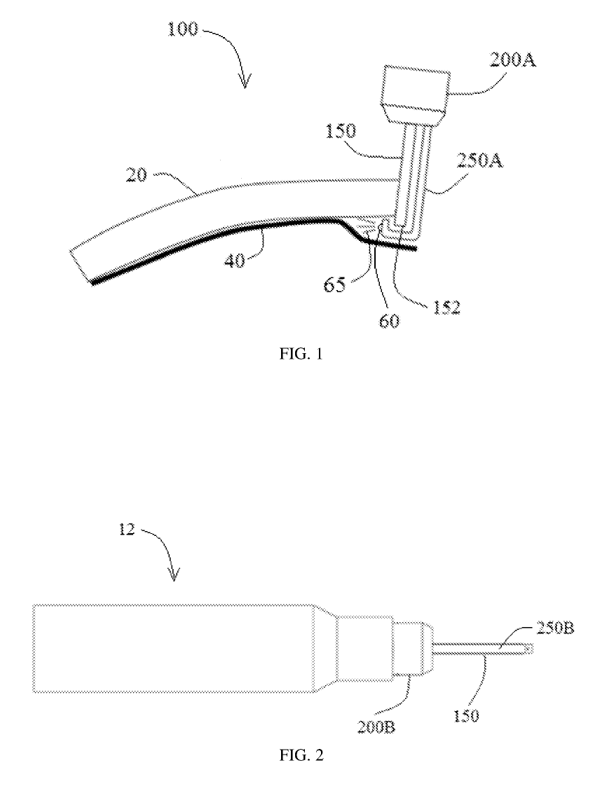

FIG. 2 illustrates a craniotome 12 including device 200B, according to one example. The view presented illustrates relative alignment as to rotary cutter 150 and guard 250B with guard member 250B shown in the foreground and cutter 150 shown in the background.

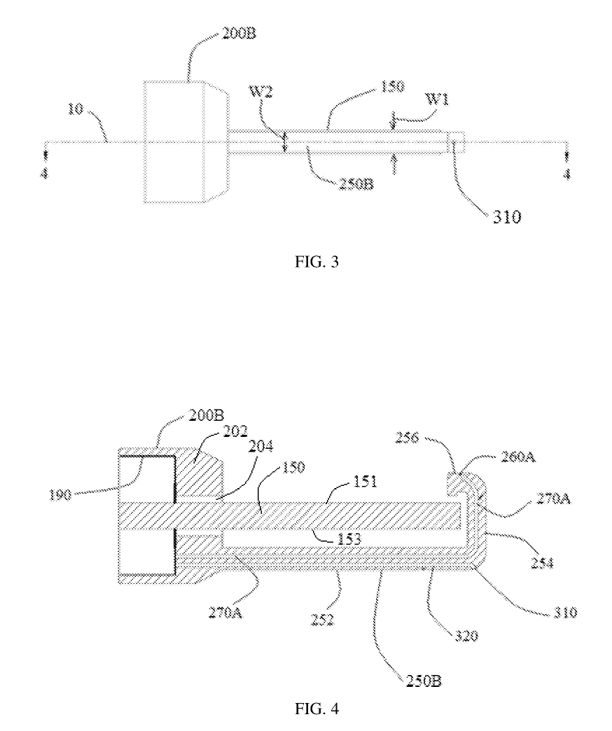

FIG. 3 illustrates a closer view of device 200B and includes suction port 310 described below. In the figure, cutter 150 has a cutting diameter or width W1 that exceeds the width W2 of guard 250B. In an example, a width of guard 250B can be between about 1 and 2 mm. The figure illustrates discharge nozzle 260A disposed on a portion of guard 250B. In the figure, discharge nozzle 260A is configured to eject fluid in a direction out of the figure and towards the reader. A cross section view along section line 4-4 is illustrated in FIG. 4. The orientation of the guard 250B relative to the cutter 150 is described further below.

FIG. 4 illustrates a cross section view of device 200B, according to one example in which the device 200B can include an adapter 202 and guard 250B. Adaptor 202 has a mating feature that aligns with an existing craniotome. Adaptor 202 can be configured such that guard 250B can be removably attached to the craniotome. In the figure, the mating feature is cavity 190, which is configured to engage with a corresponding feature of an existing craniotome. Cavity 190 can include a mechanical locking feature to positively affix device 200B onto a body diameter of a craniotome device. Cavity 190 is an example of a mating feature for engagement of the adapter 202 with the craniotome. A threaded fastener or a bayonet locking feature are examples of such mating features to removably attach adapter 202 to the craniotome. It is recognized that additional or alternative mating features can be used.

FIG. 4 illustrates relative alignment of burr 150 and guard 250B. Burr 150 can pass through an opening 204 in adaptor 202 such that burr 150 can be attached to the craniotome. Burr 150 is configured to rotate during operation of the craniotome, whereas adapter 202 and guard 250B are stationary relative to the craniotome, Guard 250B includes portions described here as leg 252, foot 254, and return 256, each of which are configured to restrict exposure of burr 150. For example, a portion of burr 150 disposed on an opposite side of leg 252 can be employed as a cutting face for conducting a craniotome procedure and other portions of burr 150 are protected from contacting tissue. In an example, foot 254 can be generally traverse to leg 252 such that a 90 degree bend connects leg 252 and foot 254 together.

Burr 150 can be described as having a working surface 151 and a non-working surface 153. Because burr 150 is rotating during operation of the craniotome, the working surface 151 can be constantly changing with rotation of burr 150. The working surface 151 can also be referred to herein as an exposed portion of the burr 150. The non-working surface 153 can be a protected portion of the burr 150 given a proximity of the leg 252 to the non-working surface 153.

During operation of the craniotome, as burr 150 advances in a lateral direction across the cranium of the patient (either front to back or side to side), the burr 150 is cutting the cranium. Because a leading edge portion of guard 250B at return 256 includes nozzle 260A, fluid can be discharged from nozzle 260A before burr 150 reaches that portion of the cranium, thus separating the dura from the cranium in advance of burr 150 cutting through the cranium. Leg 252 can thus trail burr 150 and can be proximate to non-working surface 153 and distal to return 256 having nozzle 260A.

Fluid channel 270A is disposed within leg 252 and foot 254 and terminates at discharge nozzle 260A. Fluid channel 270A can be disposed within guard 250B in the manner illustrated or can be disposed alongside guard 250B. A location of the outlet of fluid channel 270A, which coincides with a location of discharge nozzle 260A, can vary from what is shown in FIG. 4. The outlet of fluid channel 270A and placement of nozzle 260A is shown in FIG. 4 as being oriented at an acute angle relative to a cutter axis of the burr 150. FIG. 5 illustrates that the outlet of fluid channel 270B and placement of nozzle 270B can be oriented at or close to a right angle relative to the cutter axis or burr axis 10 of cutter 150.

Fluid channel 270A can be formed through the components of guard 250B such that the fluid directly contacts an interior of guard 250 as it flows there through. In another example, an aperture can be formed through guard 250B and tubing can be inserted into the aperture to create fluid channel 270A. In an example, fluid channel 270A can have a width ranging between about 0.3 mm and 1 mm. In an example, the width of fluid channel 270A can be about 0.5 mm.

Although channel 270A is shown through leg 252 as well as through foot 254 and return 256, other configurations of channel 270A can be used to deliver fluid to the dura/cranium junction, ahead of burr 150, such that the dura is removed from the cutting path of burr 150. As an example, tubing can be disposed within leg 252 and then such tubing can be exposed proximate to the working surface 151 of the burr 150 in an area distal to the leg 252.

Discharge nozzle 260A is configured to eject pressurized fluid in a pattern suited for separating the dura and cranium. Thus fluid channel 270A is fluidly connected to discharge nozzle 260A at one end and fluidly connected to a fluid supply at an opposite end. In an example, the fluid supply can include a pump, a pneumatic system, a syringe or other designs for providing a pressurized fluid into and through the fluid channel 270A to discharge nozzle 260A. The fluid supply can be external to the craniotome. A supply line from the fluid supply to the fluid channel 270A can extend through an interior of the craniotome.

In the example illustrated, vacuum channel 320 is disposed in leg 252 and terminates suction port 310 disposed near an end of leg 252. A vacuum drawn on channel 320 will allow suction port 310 to remove fluid from a cutting region during a craniotome procedure. Suction port 310 can be configured to remove the fluid ejected by discharge nozzle 260A.

FIG. 5 illustrates device 200D, according to one example. In the figure, rotary burr 150 is protected by guard 250D. Guard 250D is equipped with fluid channel 270B. Channel 270B terminates at discharge nozzle 260B. Discharge nozzle 260B is configured to eject fluid in line with discharge axis 80. Angle α between discharge axis 80 and burr axis 10 (or cutter axis 10) can be configured to provide a fluid discharge direction in accordance with the cutting conditions. For example, this can be determined based on cutter speed, cutting feed rate, extent of cut, and other factors. In one example, angle α is an acute angle and is selected to direct fluid in direction away from the dura. In an example, angle α is about equal to 90 degrees.

Angle α can be selected to reduce likelihood of directing the discharge water jet directly at brain tissue in the event that the dura tears. For example, the water stream can be angled up towards the cranium. An orientation of the discharge nozzle 260B can be used to control an angle at which the fluid contacts the tissue. Moreover, as shown in FIG. 1, it can be common during the craniotomy for the surgeon to angle the craniotome tool, relative to the cranium 20. This can also impact an angle of the fluid stream relative to the dura and cranium.

In one example, the guard can be sized and shaped to push the dura out of the way after the discharge fluid separates the dura from the cranium.

Discharge nozzle 260A can include a nozzle configuration to facilitate separation of tissue using a variety of discharge patterns. For example, the nozzle can be configured to eject water in a fan pattern, a circle or cone pattern, or a focused point shaped fluid stream.

In one example, the discharge fluid includes a gas such as compressed air.

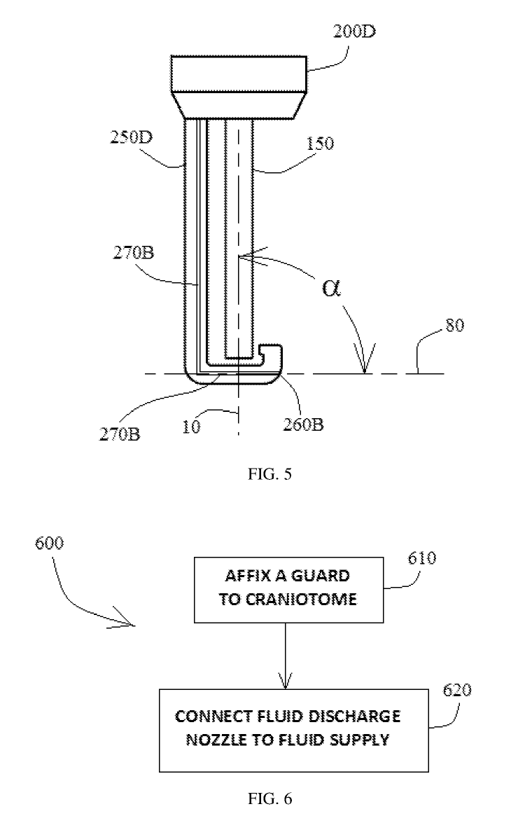

FIG. 6 illustrates method 600 associated with a craniotome, according to one example. At 610, method 600 includes affixing a guard to a craniotome. The guard can be affixed by a mechanical fastener, a clamp, a threaded fastener, or other structure. At 620, method 600 includes connecting a fluid discharge nozzle to a fluid supply. The fluid supply can be configured to provide pressurized fluid, such as water or saline. The connection can include a plumbing or tube fitting and clamp. The discharge nozzle is disposed on the guard in a manner to direct ejected fluid at a region proximate the junction of the dura and the cranium or subcutaneous tissue.

The guard of the present subject matter can be fabricated of a metal, such as stainless steel, or a plastic.

### Various Notes

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A device comprising:
an adapter configured to couple with a craniotome;
an elongate leg having a longitudinal axis, the elongate leg having a first end and a second end, the first end coupled to the adapter, wherein the longitudinal axis is aligned substantially parallel with a cutter axis of the craniotome;
a guard coupled to the second end and having a surface configured to engage with a tissue and configured to preclude contact of tissue with an end of a cutter of the craniotome; and
a fluid discharge nozzle coupled to the guard and configured to direct an ejected fluid from the guard.

2. The device of claim 1 wherein the fluid discharge nozzle has a discharge axis and wherein an included angle between the discharge axis and the cutter axis is about equal to or less than 90 degrees.

3. The device of claim 1 wherein the nozzle is configured to eject the fluid in a direction aligned radially with respect to the cutter axis.

4. The device of claim 1 wherein the fluid discharge nozzle is configured to discharge fluid in a fan pattern.

5. The device of claim 1 wherein the elongate leg has a lateral width less than a cutter width of the cutter.

6. The device of claim 1 wherein a cutting surface of the cutter is in opposition to the elongate leg.

7. The device of claim 1 wherein the fluid discharge nozzle is fluidly coupled to a fluid path disposed within the guard.

8. The device of claim 7 wherein the fluid path is disposed within at east a portion of the elongate leg.

9. The device of claim 8 wherein the fluid path is contained within a tube disposed in an interior of the elongate leg and the guard.

10. The device of claim 1 further including a suction port coupled to the guard.

11. The device of claim 10 wherein the suction port is coupled to a vacuum channel disposed within the guard and the elongate leg.

12. The device of claim 1 wherein the adapter includes an opening configured to receive the cutter such that a portion of the cutter passes through the adapter and attaches to the craniotome.

13. A device comprising:
an adaptor having a mounting surface configured to fixedly couple with a craniotome, the craniotome having a cutter;
an elongate leg having a first end coupled to the adapter and a second end distal from the craniotome, the elongate leg having a leg axis aligned substantially parallel with a rotation axis of the cutter, the second end configured to limit exposure of a contact end of the cutter; and
a fluid channel at least partially disposed within the elongate leg and coupled to a pressurized fluid source at the first end, the fluid channel coupled to a discharge port, and wherein the discharge port is configured to eject a fluid from the fluid channel;
wherein the cutter has a working surface relative to the rotation axis and a nonworking surface relative to the rotation axis, wherein the working surface and the nonworking surface are semicircular; and
wherein the discharge port is disposed proximate the working surface and distal from the non-working surface, and wherein the elongate leg is proximate the non-working surface and distal from the discharge port.

14. The device of claim 13 further comprising a foot portion coupled to the second end of the elongate leg and oriented generally traverse to the leg axis, the foot portion configured to limit an insertion depth of the cutter.

15. The device of claim 14 wherein the fluid channel is disposed within the foot portion.

16. The device of claim 13 further comprising a suction port coupled to a second channel, wherein a portion of the second channel is disposed within the elongate leg.

17. The device of claim 13 wherein the discharge port is configured to eject the fluid in a fan pattern.

18. A method of assembling a craniotome for performing a craniotomy, the method comprising:
providing a guard portion, the guard portion comprising:
an adapter having a feature for attachment to a craniotome;
a leg having a first end coupled to the adapter and a second end opposite the first end;
a foot coupled to the second end of the leg, the foot and leg oriented generally transverse to one another, the foot including a channel extending through an interior of the foot; and
a discharge nozzle coupled to the channel and configured to discharge a fluid flowing through the channel;
attaching the guard portion to the craniotome;
attaching a burr to the craniotome, the burr configured to rotate relative to the adapter during operation of the craniotome; and
orienting the burr and the guard portion relative to each other such that the leg and foot restrict exposure of the burr.

19. The method of claim 18 wherein attaching the burr to the craniotome includes passing the burr through an opening in the adapter.

20. The method of claim 18 wherein the fluid flowing through the discharge nozzle has a discharge axis, the method further comprising orienting the burr and the guard portion relative to each other such that an angle between the discharge axis and a cutter axis of the cutter is less than or equal to about 90 degrees.

21. The method of claim 18 further comprising connecting the channel in the foot to a fluid supply to deliver the fluid through the channel and to the discharge nozzle.

22. A method of performing a craniotomy on a patient, the method comprising:
moving a rotating cutting tool along a cutting path relative to a cranium of the patient;
interposing a guard between the cutting tool and a dura of the patient such that exposure of the cutting tool to the dura is restricted; and
ejecting fluid from the guard in a direction aimed at a junction of the dura and the cranium to separate the dura and the cranium and to remove the dura from the cutting path.

23. The method of claim 22 wherein the guard and the cutting tool are part of a craniotome.

24. The method of claim 23 wherein the guard includes an adapter to removably attach the guard to the craniotome.

25. The method of claim 22 wherein the guard includes an elongate leg and a foot extending from the elongate leg, the foot oriented generally transverse to the elongate leg.

26. The method of claim 22 wherein ejecting fluid from an end of the guard includes coupling a discharge nozzle to an end of the guard.

27. The method of claim 26 wherein the discharge nozzle is configured to eject the fluid at an acute angle relative to a cutter axis of the cutting tool.

28. The method of claim 22 wherein ejecting fluid from an end of the guard includes transporting fluid through a channel within an interior of the guard.

29. The method of claim 28 further comprising supplying pressurized fluid to the channel.
